(19) United States
(12) Patent Application Publication
(10) Pub. No.: US 2001/0018604 A1
(43) Pub. Date: Aug. 30, 2001

(54) Shoulder conformal therapy component of an animate body heat exchanger

(76) Inventor: William Elkins, San Jose, CA (US)

Correspondence Address: C. Michael Zimmerman, P.O. Box 730393, San Jose, CA, 95173, US

(21) Appl. No.: 09/798,261

(22) Filed: Mar. 1, 2001

Related U.S. Application Data
(63) Continuation-in-part of application No. 09/173,637, filed on Oct. 16, 1998.

(51) Int. Cl. A61F 7/00

(52) U.S. Cl. 607/108

(57) ABSTRACT

A shoulder component is described for an animate body heat exchanger, which shoulder component has both a compliant bladder for confining a heat exchange medium and a gas pressure bladder which overlies such heat exchange bladder. The shoulder component includes a harness arrangement which connects to a standard waist belt to facilitate maintaining such shoulder component in position on the shoulder of a wearer. It also includes a alteration adjustment in the form of a flap and relief to enable minor configuration adjustments as necessary to assure good thermal contact.

### CROSS REFERENCE TO RELATED APPLICATIONS

This patent application is a continuation-in-part of patent application Ser. No. 09/173,637 filed Oct. 16, 1998 and entitled COMPLIANT HEAT EXCHANGE SPLINT AND CONTROL UNIT, naming the inventor hereof as a co-inventor.

### DISCLOSURE

### Background of the Invention

The present invention relates to a conformal therapy component of an animate body heat exchanger and, more particularly, to such a component which is particularly designed to assure both that it stays in place and also achieves good thermal conduction with a portion of an animate body to be treated.

It is now common to apply cold and compression to a traumatized area of a human body to facilitate healing and to prevent unwanted consequences of the trauma. In fact, the acronym RICE (Rest, Ice, Compression, and Elevation) is now used by many.

Cold packing with ice bags or the like has traditionally been used to provide deep core cooling of a body part. Elastic wraps are often applied to provide compression.

It will be appreciated that the traditional techniques are quite uncontrollable. For example, the temperature of an ice pack will, of course, change when the ice melts; and it has been shown that the application of elastic wraps and, consequently, the pressure provided by the same, varies considerably with differing wrappers even when the wrapping is done by experienced individuals.

Because of these difficulties and for other reasons, many in the field have turned to more complicated animate body heat exchangers. An effective animate body heat exchanger includes two major components: (1) an external conformal therapy component for covering a body portion to be subjected to heat exchange and compression; and (2) a control unit for producing a flowing heat exchange fluid and a pressurized gas. The external conformal therapy component should have two bladders, a first one of which confines a heat exchange liquid or other medium and a second of which overlays the first and confines gas pressure to be applied to the body portion to inhibit edema and to apply pressure against the heat exchange liquid bladder to press it toward the body portion. It should be noted that most animate body heat exchangers available at the time of filing do not have two bladder arrangements, nor a control unit which provides both pressurized air and a flowing liquid.

It is important that a conformal therapy component be configured and maintained in good thermal conductive relationship with the body portion to be treated. One of the difficulties is that the configuration of the animate body portion to be treated may differ among differing individuals. For example, the shoulders of humans often differ among individuals—some humans have somewhat sloping shoulders whereas others have relatively non-sloping shoulders, and some have wide shoulders and some have narrow shoulders. It therefore will be recognized that it is difficult to provide a generic conformal therapy component designed for all of the individuals of a species.

One other problem relating to thermal conduction is that many body portions are not generally suited to facilitate maintaining a therapy component in a particular place for the full time of a desired treatment. That is, the therapy component may tend to move around and, consequently, this deleteriously affects its ability to provide thermal conduction and/or compression.

### SUMMARY OF THE INVENTION

The present invention addresses the above problems. It includes a configuration altering adjustment to enable one to alter the configuration of the component or its bladder(s) to fit individual wearers. That is, a user of the component is able to make it accommodate the variations found among individuals. It further includes a connector designed to engage a separate connection positionable on the animate body so as to aid in holding the component in place.

Most desirably, the configuration altering adjustment has two parts, a first one of which is designed to maintain a desired configuration alteration and a second one of which is designed to facilitate achieving such desired alteration. More simply, the first part is a flap for selectively connecting two parts or aspects of the therapy component together, and the second part is a relief defined by the perimeter configuration of the component itself. In a preferred embodiment, the conformal therapy component of the invention is designed for treating the shoulder of a human being, and the connector extends from the shoulder to the opposite side of the human to engage a belt at the waist of the individual being treated.

Other features and advantages of the invention either will become apparent or will be described in connection with the following, more detailed description of a preferred embodiment of the invention and variations.

### BRIEF DESCRIPTION OF THE DRAWINGS

With reference to the accompanying two sheets of drawing:

FIG. 1 is a front elevation view of a human, showing in position a preferred embodiment of a shoulder conformal therapy component incorporating the invention;

FIG. 2 is a rear elevation view of the individual of FIG. 1, showing the opposite side of the therapy component incorporating the invention;

FIGS. 3A and 3B are broken-away side elevation views showing two different configuration alterations provided by the preferred embodiment of the invention; and

FIG. 4 is a planer layout of many aspects of the conformal therapy component of the invention.

### DETAILED DESCRIPTION OF A PREFERRED EMBODIMENT

The following, relatively detailed description is provided to satisfy the patent statutes. It will be appreciated by those skilled in the art, though, that various changes and modifications can be made without departing from the invention.

A shoulder therapy component is generally referred to in the figures by the reference numeral 11. This conformal component is configured to fit the typical shape of a shoulder of a human as is illustrated. (It is shown on the human's left shoulder. When it is on the right shoulder the flap to be discussed will face rearward instead of frontward.)

As will be described in more detail hereinafter, shoulder component 11 includes a compliant bladder designed to confine a heat exchange medium, typically a liquid. The component and such bladder is configured to fit the typical shape of a shoulder in good conductive relationship thereto. It must be remembered, though, that shoulders have a configuration which varies somewhat among different individuals. Thus, while shoulder therapy component 11 has a configuration to fit the typical shape of a human shoulder in good thermal conduction relationship, minor adjustments in configuration are needed to assure that a good thermal relationship with an individual shoulder is achieved.

Shoulder component 11 is one component of an animate body heat exchanger which also includes a control unit 12. Such shoulder component is connected to the control unit via tubes as will be discussed. Tubes 13 and 14 extend all the way to the control unit and are for circulating a temperature controlled liquid to the compliant liquid bladder, whereas tubes 15 and 16 join together before the control unit and deliver and relieve gas pressure to a gas bladder (also to be discussed in more detail hereinafter) which is part of the component 11, overlaying the liquid bladder. Such tubes are located within, and protected by, a sleeve 17 extending from the control unit to adjacent (though not shown) the shoulder component. Sleeve 17 is often referred to as a “umbilical” cord since it represents the operative connection of the control unit to the conformal therapy component 11.

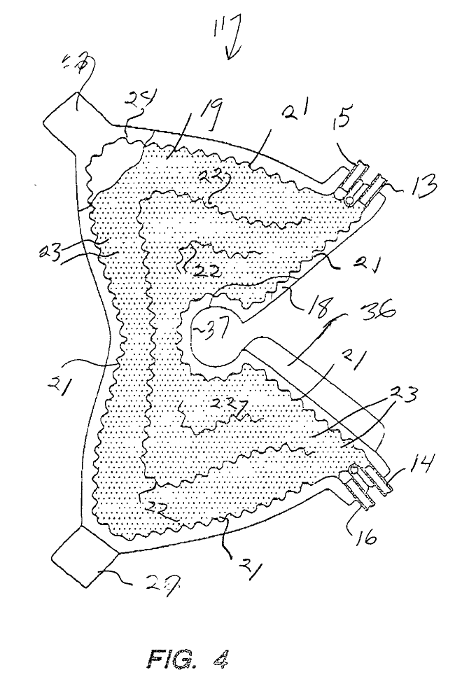

Before the particular manner in which the shoulder component is positioned and maintained in place is described, details as to some of the operative aspects of such component should be understood. One of these operative aspects is the compliant bladder designed to confine a heat exchange medium. With reference to FIG. 4, it will be seen that such bladder is defined by two walls formed from flexible sheets 18 and 19 of a heat sealable material. These two sheets form the outer and inner flexible walls of such bladder. (Although the wall formed by the sheet 18 is an “outer” wall insofar as the component itself is concerned, it is the wall which is the closest described to the shoulder of the individual undergoing treatment, i.e., an “inner” wall relative to the shoulder.)

The sheets 18 and 19 forming walls of the heat exchange medium bladder are secured together by a heat sealing process to form a curvilinear border 21. This border 21 is formed by a plurality of curvilinear ripples which generally are significantly shorter in length than the border portion of which each is a part. Such curvilinear ripple construction, among other things, prevents eddies from forming during liquid flow through the bladder. (The terminology “curvilinear border” as used herein means that position of the heat seal at the edge of the bladder which is exposed to liquid flow in the bladder—the configuration of the heat seal that is not exposed to such liquid flow is irrelevant to such flow.) In this connection, while from the broad standpoint the heat exchange medium could be any appropriate substance, it is preferred that it be a temperature controlled liquid circulated by the control unit. Such liquid passes into the compliant bladder through tube 13 and exits from such bladder through tube 14. The temperature control agent is a flowing liquid because, among other reasons, of a liquid's thermal capacity and its thermal response time—an important capability because of the desire to change the thermal capacity of the medium at various locations, i.e., by replacing heat exchange medium at a location which already has transferred thermal energy with the body portion.

In accordance with patent application Ser. No. 09/127,256 mentioned above, fences or dividers 22 are provided within the heat exchange liquid bladder to aid in defining liquid flow therethrough. These fences 22 are formed during the heat sealing process and have the same curvilinear construction as the border 21. The interior of the bladder also includes a pattern of dots 23 or, in other words, a dot matrix which disperses the liquid during its flow through the conformal bladder defined by the walls 18 and 19. (It should be noted that the pattern formed by the dots 23 and the fences 22 is apparent from the exterior side of wall 18.)

As mentioned previously, the flexible sheets of material from which the walls 18 and 19 are formed are heat sealable together to form the curvilinear border 21, the fences 22, and the dots 23. The sheets of material providing these walls in one realization of the invention, nylon, coated with polyurethane to provide the same with the heat sealing qualities and the impermeability to liquid needed.

The shoulder component also includes a compliant gas pressure bladder overlying the heat exchange medium bladder to direct gas against the shoulder and press the heat exchange bladder toward such shoulder. This bladder simply is provided by adding an additional sheet 24 to the sandwich making up the operative aspects of the component being described. It cooperates with the sheet 19 to define such bladder. That is, sheet 19 does “double duty.” One side of the same provides a wall for the heat exchange medium bladder, and the other side provides a wall for the gas pressure bladder.

While the fences 22 are “telegraphed” through to the gas bladder in order to maintain the sheets 24 and 19 relatively close together, the dot matrix is not in the particular realization of the invention mentioned above. It should be noted, though, that only pieces of the fence and/or for the dot matrix may be provided in the gas bladder as desired since it (or their) purpose is to maintain the two sheets together in order to control “ballooning” rather than to direct flow.

The sheet 24 is of the same polyurethane coated nylon as the sheets 18 and 19, and all three sheets are heat sealed together, i.e., the border 21 is common to both bladders with the result that the conformal gas pressure bladder registers with the conformal heat exchange liquid bladder. However, the sheet of material 24 is a composite sheet with an outer layer 26 made of a loop fastening material of a fastener, such as of the Velcro™-type.

In one realization of the invention, a cyclic air pressure was introduced into the gas pressure bladder, which pressure cycled between about 0.25 psig and about 1.5 psig every two minutes or so. (It should be noted that the provision of both of the tubes 15 and 16 provides a faster response time to the cycling.) The thermally controlled flowing liquid was maintained at about 45° F. in this realization, to cool the shoulder of the wearer. It was a 20 percent propylene glycol solution of distilled water, with a small amount of both an antifungicide such as iodine and a non-foaming wetting agent to break surface tension. The flow rate was about 40-60 pounds/hour with a liquid pressure of between about 5 and 20 psig. The control unit used in such realization included an ice bath surrounding a container through which the liquid was circulated after being returned from the heat exchange bladder. (It should be noted that this control unit was capable of supplying liquid at other controlled temperatures.)

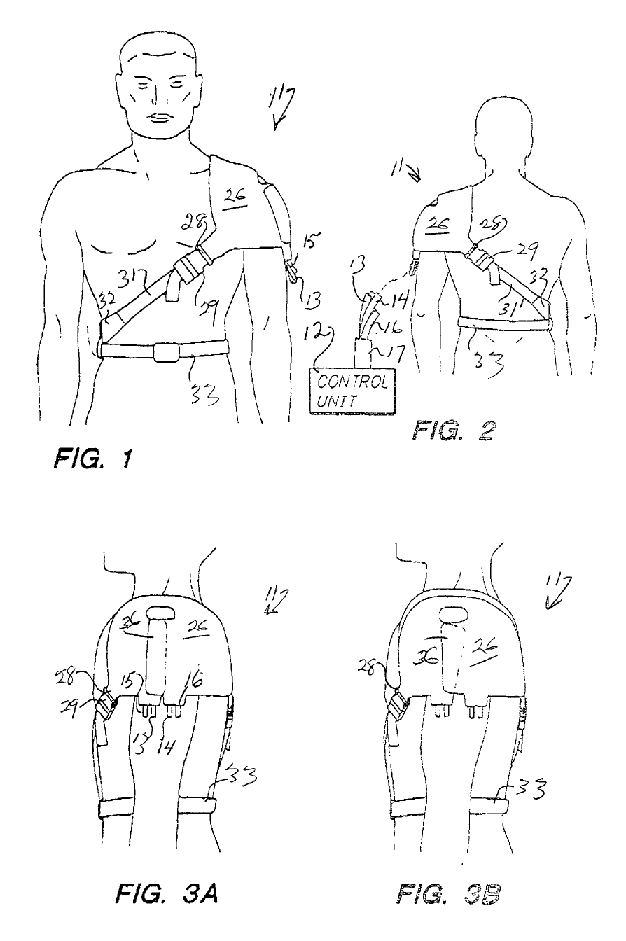

The flexible sheets 24 form a pair of tab extensions 27 which facilitate the connection thereto via respective elongated rings 28, of quick disconnect buckles 29 (see FIGS. 1 and 2).

As illustrated, these buckles are on both sides of the shoulder component and enable the securance thereto of straps 31 and 31′. As shown, each of these straps interacts with it respective disconnect buckle to enable adjustment. Moreover, the other end of each of these straps is respectively connected to associated ends of a loop fastening strap 32. As is shown, the strap 32 encircles a separate connection in the form of a belt 33 placed about the waist of the wearer of the component. The end of the strap 32 is provided with a tab of hook material so that after encircling the belt, the strap can engage itself, i.e., the engaging parts of the strap have complementary hook and loop fastening surfaces. To facilitate an understanding, FIG. 1 shows the engaging part which encircles the belt in both a phantom position before it is engaged with the remainder of the strap 32, and a solid position in which it is so engaged: FIG. 2 only shows the strap engaged.

It will be recognized that such an engagement of a conformal therapy component with a separate connection can play a major role in holding a therapy component in place. This is particularly true with the shoulder therapy component of the preferred embodiment. The connection maintains the shoulder component in good thermal relationship with the shoulder of the wearer.

As mentioned previously, the configuration of human shoulders and other animate body portions vary somewhat between differing individuals. This can interfere with the desired good thermal relationship desired between the therapy component and the body portion even when, as is the case here, the thermal component is configured to fit the typical shape of the animate body portion. As another aspect of the invention, the component also includes a configuration altering adjustment. This configuration altering adjustment is positioned relative to the compliant heat exchange bladder to enable one to alter the configuration of the bladder as desired, e.g., to fit the shape of the shoulder of the particular individual undergoing treatment. One is also able to use it to secure the component to the individual on which such component is installed.

In this preferred arrangement, the altering adjustment has two parts: a first part designed to maintain a desired configuration alteration, and a second part designed to facilitate achieving the desired alteration.

These two parts and their use is quite simply achieved. The first part is simply a flap 36 which is part of the sheet 24 and extends outward from one edge of such component. Flap 36 is provided with the hook portion of a Velcro™-type fastening mechanism and therefore can interact with the outer surface 26 of the component to maintain a desired altered configuration. The second part in this embodiment is simply a relief 37 in the perimeter of the component and its constituent bladders. As can best be understood by comparing FIGS. 3A and 3B, this relief facilitates altered adjustment with the flap by enabling the same while inhibiting undesired changes in configuration. Use of the flap enables one to alter the configuration of the bladder infinitely through a limited range. For example, as is shown in FIG. 3B, the interaction between the flap and the remainder of the component might be a “canted” one in order to accommodate varying shoulder slope and to obtain the desired good thermal relationship. It should be noted that the adjustment as described is associated with both the heat exchange medium and the gas pressure bladders.

As mentioned at the beginning of the detailed description, applicant is not limited to the specific embodiment and variations described above. They are exemplary, rather than exhaustive. The claims, their equivalents and their equivalent language define the scope of protection.

1. A conformal therapy component of an animate body heat exchanger for applying a desired treatment to an animate body portion that has a configuration which varies somewhat among differing individuals, said therapy component comprising the combination of:
A. a compliant bladder designed to confine a heat exchange medium, configured to fit the typical shape of such an animate body portion; and
B. a configuration altering adjustment associated with said compliant bladder and positioned relative thereto to enable one to alter the configuration of said bladder as desired:

2. The conformal therapy component of claim 1 wherein said configuration altering adjustment also is able to secure said component to said body part of an individual on which it is installed.

3. The conformal therapy component of claim 1 wherein said adjustment is designed to enable the configuration of said bladder to be altered as necessary to fit the shape of an animate body portion of an individual in good thermal conductive relationship thereto.

4. The conformal therapy component of claim 1 further including as part of said combination, a compliant gas pressure bladder overlaying at least a portion of the heat exchange medium bladder to direct gas pressure against said heat exchange medium bladder and press the same toward said body portion, said configuration altering adjustment being associated with both of said compliant bladders and positioned relative to the same to enable one to alter the configuration together of said bladders as desired.

5. The conformal therapy component of claim 1 wherein said adjustment is designed to enable one to alter the configuration of said bladder infinitely through a limited range.

6. The conformal therapy component of claim 1 wherein said adjustment has two parts, a first one of which is designed to maintain a desired configuration alteration and a second one of which is designed to facilitate achieving a desired alteration.

7. The conformal therapy component of claim 6 wherein said second part is a relief defined by the perimeter configuration of said conformal therapy component.

8. The conformal therapy component of claim 6 wherein said first part is a flap for selectively connecting two aspects of the remaining portion of the component together.

9. The conformal therapy component of claim 8 wherein said flap includes a loop or hook type of fastening surface positioned to interact with a complementary hook or loop fastening surface on the remainder of said therapy component.

10. The conformal therapy component of claim 9 in which said compliant bladder is configured to fit the typical shape of a human shoulder.

11. A conformal therapy component of an animate body heat exchanger for applying a desired treatment to an animate body part that has a configuration which varies somewhat between individuals, said therapy component comprising the combination of:
A. a compliant bladder designed to confine a heat exchange medium, configured to fit the typical shape of such an animate body portion in good thermal conductive relationship thereto; and
B. a connector as part of said therapy component designed to engage a separate connection positionable on said animate body, to aid in holding said therapy component in place at said body portion.

12. The conformal therapy component of claim 11 further including a configuration altering adjustment associated with said compliant bladder and positioned relative thereto to enable one to alter the configuration of said bladder to fit differing shapes of said animate body portion.

13. The conformal therapy component of claim 11 wherein said connector is designed to maintain said compliant bladder in good thermally conductive relationship with said body portion when it engages said separate connection.

14. The conformal therapy component of claim 14 wherein said connector is a strap.

15. The conformal therapy component of claim 14 wherein said component is designed to apply a desired treatment to the shoulder of an individual, and said separate connection is a belt designed to be placed about the waist of a wearer.

16. The conformal therapy component of claim 15 in which said strap is configured to encircle said belt.

17. The conformal therapy component of claim 16 wherein said strap is configured to engage itself after encircling said belt, the engaging parts of said strap having complementary hook and loop fastening surfaces for maintaining said engagement.

18. A conformal therapy component of an animate body heat exchanger for applying a desired treatment to an animate body portion that has a configuration which varies somewhat among differing individuals, said therapy component comprising the combination of:
A. a compliant bladder designed to confine a heat exchange medium, configured to fit the typical shape of such an animate body portion in good thermal conductive relationship thereto;
B. a configuration altering adjustment associated with said compliant bladder and positioned relative thereto to enable one to alter the configuration of said bladder as desired, said adjustment being designed to enable the configuration of said bladder to be altered as necessary to fit the shape of an animate body portion of an individual and having two parts, a first one of which is designed to maintain a desired configuration alteration and a second one of which is designed to facilitate achieving a desired alteration aspects of said component.
C. a compliant gas pressure bladder overlaying at least a portion of the heat exchange medium bladder to direct gas pressure against said heat exchange medium bladder and press the same toward said body portion, said configuration altering adjustment being associated with both of said compliant bladders and positioned relative to the same to enable one to alter the configuration together of said bladders as desired;
D. a connector as part of said component designed to engage a separate connection positionable on said animate body, to aid in holding said component in place at said body portion, said connector being designed to maintain said compliant bladder in good thermally conductive relationship with said body portion when it engages said separate connector.

19. The conformal therapy component of claim 18 wherein said connector is a strap.

20. The conformal therapy component of claim 19 wherein said component is designed to apply a desired treatment to the shoulder of an individual, and said separate connection is a belt designed to be placed about the waist of a wearer.
